(19) United States
(12) Patent Application Publication
(10) Pub. No.: US 2005/0266001 A1
(43) Pub. Date: Dec. 1, 2005

(54) Therapeutic blockade of CD103 interactions to prevent clinical renal allograft rejection

(76) Inventor: Gregg Allen Hadley, Phoenix, MD (US)

Correspondence Address: BUCHANAN INGERSOLL PC;(INCLUDING BURNS, DOANE, SWECKER & MATHIS), POST OFFICE BOX 1404, ALEXANDRIA, VA, 22313-1404, US

(21) Appl. No.: 11/097,125

(22) Filed: Apr. 4, 2005

Related U.S. Application Data
(60) Provisional application No. 60/559,255, filed on Apr. 2, 2004.

(51) Int. Cl. A61K 39/395

(52) U.S. Cl. 424/144.1

(57) ABSTRACT

Methods of preventing or treating allograft rejection, particularly clinical renal allograft rejection, comprising administering to a patient in need thereof a therapeutically-effective amount of a composition which affects the expression, activity, or other function of CD103 are disclosed. Also disclosed are methods for identifying agents which modulate the activity or expression of a CD103 protein and animal, particularly mouse, models of renal allograft rejection.

### FIELD OF THE INVENTION

The present invention relates to methods of preventing clinical renal allograft rejection.

### BACKGROUND OF THE INVENTION

Despite the routine nature of clinical renal transplantation, even well-matched transplants are recognized and inevitably destroyed by the cellular arm of the adaptive immune system (1) Therapeutic strategies to counter this process continue to rely on drugs that globally suppress the adaptive immune system, leaving the patient vulnerable to tumors and opportunistic infections. Recent advances in immunosuppressive strategies have led to a progressive improvement in short term renal allograft survival (2). Nonetheless, even in the modern era of immunosuppression, the half-life of cadaveric kidney transplants that survive the first year is still quite modest, ranging from 6-19 years depending on the degree of HLA match (1). Consequently, late graft loss now represents the major rejection problem in clinical renal transplantation. Although overt cellular rejection episodes occurring in the first few months post-transplantation are increasingly rare, there is now compelling evidence that such events inflict lasting damage to the graft which manifests as an increased risk of late graft loss (3). The present invention is useful for preventing clinical renal allograft rejections. Alternatively, the present invention provides a decrease in the risk of late graft loss.

### SUMMARY OF THE INVENTION

The present invention relates to methods of preventing clinical renal allograft rejection. The prevention may include a decrease in whole or in part of CD103 expression, enzymatic activity, or other function. The present invention further relates to methods of identifying compounds useful in the treatment and prevention of clinical renal allograft rejection. The present invention also relates to compounds useful in the treatment and prevention of clinical renal allograft rejection. The present invention still further relates to the use of compounds that alter TGF-beta receptor expression or activity in the treatment and/or prevention of clinical renal allograft rejection. The present invention also relates to the use of compounds that alter CD103 expression or activity in the treatment and/or prevention of clinical renal allograft rejection. “Alter” is meant to include an increase or decrease in whole or in part of CD103 expression or activity. The present invention also relates to antibodies to CD103. The present invention further relates to antibodies to TGF-beta receptor. The present invention also relates to the use of these antibodies to treat and/or prevent clinical renal allograft rejection. The present invention still further relates to a clinical animal model of renal allograft rejection.

### BRIEF DESCRIPTION OF THE DRAWINGS

This patent or application file contains at least one photograph executed in color. Copies of this patent or patent application publication with color photographs will be provided by the Office upon request and payment of the necessary fee.

FIG. 1A shows the serum creatinine concentration per day for each renal allograft, unmodified, as well as histological analyses. FIG. 1B shows the serum creatinine concentration per day for each renal allograft, CsA delayed, as well as histological analyses. The abrupt rise in serum creatinine indicates that the renal allografts in this strain combination were rapidly rejected.

FIG. 2A, FIG. 2B, and FIG. 2C show fluorescence activated cell sorting (“FACS”) analyses indicating CD103 expression in: FIG. 2A—various CD8 cells that infiltrated allografts; FIG. 2B—lymphocytes that infiltrated renal allografts undergoing CsA-delayed rejection; FIG. 2C—CD8 cells that infiltrated renal isografts or vascularized heart allografts. FIG. 2D illustrates the absolute number of CD103+CD8+ cells in the graft steadily increased from the time of CsA withdrawal through ˜8 weeks then declined sharply by 12 weeks post-transplantation.

FIG. 3A indicates the distribution of cells in the graft that were CD103+, and CD8+ or non-CD8 cells. FIG. 3B indicates the distribution of cells in the renal lymph node of renal allograft recipients that were CD103+, and CD8+ or non-CD8 cells.

FIG. 4A shows that CD103+CD8+ cells that infiltrate renal allografts were CD44^{hi}, TCR-αβ⁺, CD11a^{hi}, CD62L^{lo}, and thus exhibit a classic CD8 T effector phenotype. FIG. 4B shows a subset of CD103+CD8+ effectors intimately associated with the renal tubular epithelium which stains brightly for E-cadherin.

FIG. 5 illustrates the abundance of CD103+CD8+ effectors correlated closely (R=0.82) with the progression of graft destruction.

FIG. 6A shows the histology indicating the levels of tubulitis and tubular destruction of rejection in control rats, and FIG. 6B shows the histology indicating the levels of tubulitis and tubular destruction of rejection in rats treated with OX-62. FIG. 6C, FIG. 6D, and FIG. 6E illustrate that OX-62 dramatically reduced accumulation of CD8+ T cells in the graft renal tubules.

FIG. 7 shows an amino acid sequence of Human CD103 (SEQ ID NO:1).

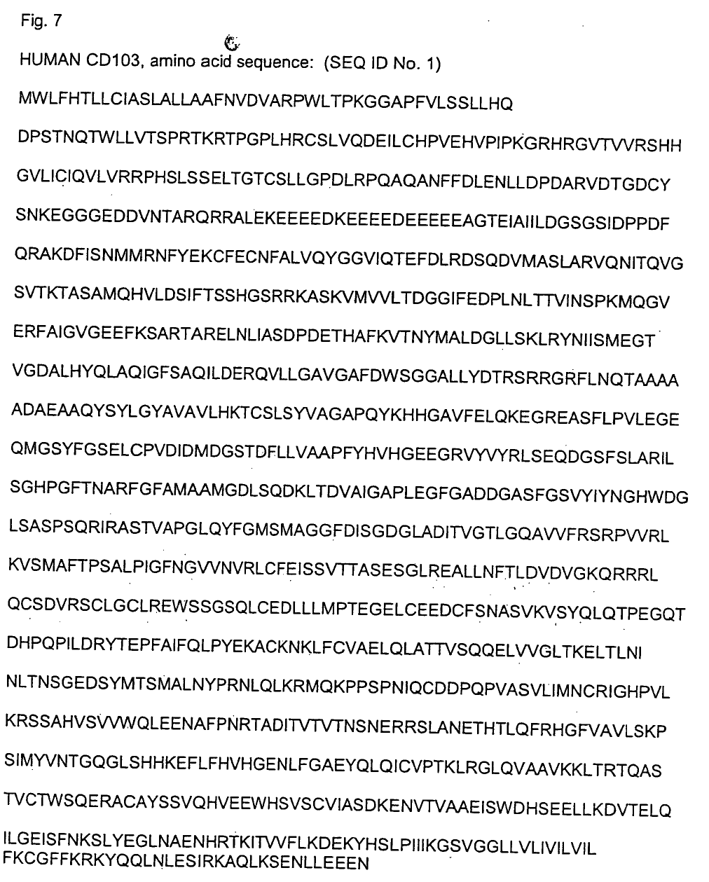

FIG. 8 shows an amino acid sequence of Mouse CD103 (SEQ ID NO:2).

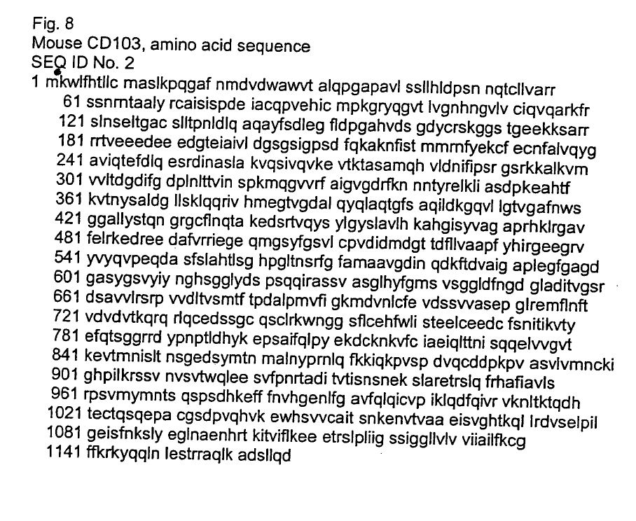

FIG. 9 shows in vitro efficacy of OX62-SAP by showing the total number of CD8+ cells in each culture following addition of OX62-SAP to CD103+CD8+ cells in culture as determined by FACS.

FIG. 10 shows representative FACS plots for the in vitro OX62-SAP study.

FIG. 11 shows the in vivo efficacy of OX62-SAP by showing the total number of CD8+ CD3+cells remaining in the intestinal intraepithelial lymphocyte (iIEL) compartment following intravenous injection of OX62-SAP in normal rats

### DETAILED DESCRIPTION OF ILLUSTRATIVE EMBODIMENTS

Role of CD8+ T-Cells in Renal Allograft Rejection

CD8+ T cells are problematic following allogeneic tissue transplantation because they cross-react at high frequency with highly-polymorphic variants of MHC class I molecules, and also recognize polymorphic peptides derived from non-MHC proteins (i.e., minor H antigens) that differ between donor and recipient in the context of self MHC class I molecules. Consequently, even well-matched allografts elicit robust CD8 responses. Ubiquitous expression of class I molecules assures that all graft cell-types are susceptible to destruction during the effector phase of the response.

Naïve CD8+ T cells are confined to lymphoid recirculation in lieu of further differentiation signals (9). However, following encounter with APC carrying donor alloantigens in secondary lymphoid compartments, donor-responsive CD8 cells initiate a program of gene expression culminating in their differentiation to T effector status (10). The salient events in the transition of naive CD8 cells to effector status include a loss of the lymph node homing receptor, CD62L, upregulation of LFA-1 and VLA-4, and alterations in chemokine receptor expression. These changes in cell surface phenotype collectively serve to release recently-activated CD8 cells from lymphoid recirculation and promote transmigration into inflammatory sites (5). Critically, recently-activated CD8 cells also initiate synthesis of diverse effector molecules that facilitate rapid destruction of cells expressing the target alloantigen. The best characterized of these effector pathways are the cytotoxic granule and Fas/FasL pathways (11), but CD8 effectors also produce cytokines (12, 13) and chemokines (14, 15) that may indirectly promote target destruction and/or recruit additional effector populations to the site.

That CD8 effector populations are key contributors to the efferent phase of clinical renal allograft rejection is supported by several lines of observational evidence. For one, CD8 effector gene expression by T cells in peripheral blood (16-20) or urine (21) of renal allograft recipients correlates closely with occurrence of rejection episodes. Furthermore, CD8+ T cells expressing specific markers of CD8 effector function infiltrate the renal tubules at the time of rejection (22, 23). These data provide compelling support for the concept that CD8 cells are centrally involved in destruction of the graft renal tubules, a long-recognized hallmark of acute renal allograft rejection (24).

Despite broad acceptance that CD8 effector populations play a decisive role in clinical renal allograft rejection, experimental data from animal models to definitively document such a role are lacking. This ambiguity exists, in large part, because of the inherent redundancy of effector mechanisms in the efferent phase of renal allograft rejection. Thus, multiple cell populations contribute to graft destruction, and those cell populations include not only CD8+ T cells, but also CD4+ T cells, macrophages, and a plethora of other leukocyte lineages (25). This redundancy confounds investigations of CD8-dependent damage to the graft. For example, CD8 blockade often fails to prevent renal allograft rejection, but it is not clear whether this reflects an actual lack of CD8 involvement or, alternatively, the additional involvement of non-CD8 effector mechanisms. The often strict requirement for CD4 help in generating and/or maintaining effective CD8 effector responses (26) introduces an additional confounding variable. Thus, blockade of the CD4 response often completely abrogates rejection but it is not clear whether this reflects a requirement for CD4 cells in providing help for CD8 effector responses, or conversely, a key role for CD4 cells in the effector phase of the response. These limitations are further compounded by the lack of suitable animal models that replicate the salient events of clinical renal allograft rejection: In the mouse, even fully MHC disparate renal allografts often fail to reject (27-30). Rat renal allografts undergo a normal tempo of rejection, but the graft histology and mechanisms of rejection are distinct from the clinical situation with an aberrant predominance of CD4+ T cells and antibody in the absence of cardinal features of clinical renal allograft rejection such as preferential migration of CD8+ T cells into the renal tubules (31).

Properties of the T-Cell Integrin CD103

Integrin family heterodimers play diverse and redundant roles in T-cell activation, homing, and delivery of effector function (32). The CD103 integrin heterodimer was initially identified by its expression on T cells in the vertebrate gut mucosa (33), where it is expressed at high levels by >95% of intestinal intraepithelial lymphocytes (iIEL) and ˜40% lamina propria lymphocytes. The studies of Karecla et al. (34) and Cepek et al. (35) established that CD103 recognizes the epithelial cell-specific ligand, E-cadherin. In normal mice and humans, CD8+ T cells that reside within the gut epithelium express high levels of CD103. Consequently, early investigations of CD103 were biased towards documenting its seemingly obvious role in promoting retention of T cells in mucosal layers where they could serve an initial line of defense against microbial attack. Surprisingly, however, CD103 knockout mice (targeted disruption of the αE gene) exhibit only a mild and inconsistent deficiency of T cells within the intestinal epithelium (36). Indeed, CD103 knockout mice are phenotypically indistinguishable from their wild type cohorts. Moreover, Lefrancois et al. (37) demonstrated that CD103 expression is not required for long-term retention of antigen-specific CD8 effector/memory T cells within the intestinal epithelium.

Unlike better known T-cell integrins such as LFA-1 (αLβ2) and VLA-4 (α4β1), CD103 is poorly expressed by peripheral T cells. In humans, CD103 expression is confined to small subset (<1%) of circulating memory T cells (38). In mice, CD103 is expressed at low levels (5-10 fold less than that of iIEL) by 40-60% of peripheral CD8 cells, the frequency of which is highly strain-dependent (39, 40). Also unlike other T-cell integrins, T-cell expression of CD103 is not invariably associated with T-cell activation. In the mouse system, CD103 expression by peripheral CD8 cells is confined to cells of naïve phenotype with little expression by memory or recently activated CD8 cells (40). However, CD103 is rapidly induced on mouse peripheral T cells following activation and subsequent exposure to TGF-β (39). Expression of CD103 on activated human T cells is similarly dependent on TGF-β (42). A recent study demonstrated that TGF-β induces rapid transcription of the αE gene (43), but the control elements responsive to TGF-β do not reside in the proximal promoter and have yet to be identified (43). The CD103 beta chain (β7 integrin) is widely expressed by T cells (44) and therefore is not thought to be a limiting factor in induction of CD103 expression by TGF-β.

Role of CD103 in Renal Allograft Rejection

FACS analyses of transplant nephrectomy specimens revealed that a major subset of CD8 effectors that infiltrated allografts undergoing rejection episodes expressed high levels of CD103 (42, 45). Robertson et al. (46) used transplant biopsy specimens to confirm that CD103+CD8+ cells are present in renal allografts during episodes of clinical dysfunction and, moreover, that such cells preferentially reside within the renal tubular epithelium. The latter study further demonstrated that CD103 is poorly expressed by CD8+ T cells in non-invasive infiltrates (i.e., those in which the severity of tubulitis is low) (46). Interestingly, CD103+CD8+ effectors are most abundant in renal allografts undergoing rejection episodes in the context of chronic allograft nephropathy (45). Importantly, CD103+CD8+ effectors are not present in peripheral lymphoid compartments (i.e., peripheral blood lymphocytes) (40), and thus are not detectable by conventional immune monitoring approaches. However, Ding et al. have shown that CD103 mRNA is expressed by cells isolated from the urine of renal allograft recipients concomitant with clinical rejection (48), consistent with the intratubular localization of CD103+CD8+ effectors during rejection episodes.

The clinical observations noted above are consistent with a key role for CD103 in promoting destruction of graft epithelial compartments by CD8 effector populations. Experimental support for this hypothesis was obtained using a mouse model of pancreatic islet transplantation (49). Similar to the renal tubules, pancreatic islets are specialized epithelial layers expressing high levels of E-cadherin, and are known to be susceptible to destruction by CD8 effector populations. Wild-type hosts uniformly reject islet allografts transplanted into the renal subcapsule, concomitant with the appearance of CD8+ CD103+effectors at the graft site (49). Strikingly, however, the majority of CD103 knockout mice accepted islet allografts indefinitely. Moreover, adoptive transfer of wild-type, but not CD103−/− CD8 cells into CD103−/− hosts with long surviving allografts elicited prompt rejection. These data provide support for the hypothesis that CD103 expression is required for CD8-mediated destruction of graft epithelial elements.

These studies related to non-vascularized islet grafts transplanted into the renal subcapsular site. However, it was not known if CD103 plays an analogous role in the destruction of the renal tubules during rejection of vascularized renal allografts. This was a critical gap in the knowledge because the immune response to vascularized renal allografts differs in several key respects from the immune response to non-vascularized islet allografts. Understanding if CD103 plays an analogous role in the destruction of the renal tubules during rejection of vascularized renal allografts is critical for utilizing methods of modulating CD103 in the prevention or treatment of clinical renal allograft rejection.

Differences Between Immune Response to Vascularized Renal Allografts vs. Non-Vascularized Islet Allografts

The differences between the immune response to vascularized renal allografts vs. non-vascularized islet allografts include a) the nature of the originating tissue (islet cells vs. kidney), b) the nature of the target (renal subcapsular space vs. kidney) c) the graft vasculature (non-vascularized vs. vascularized), among other differences. Any or all of these differences have the potential to alter the nature of the lymphocyte subsets that infiltrate rejecting allografts, and thus the relative contribution of CD103+CD8+ effectors to rejection events. Additionally, the local environment of the graft site can have a profound impact on the generation of CD103+CD8+ effector populations. The local milieu of islet allografts is not predictive of that of renal allografts.

The nature of the originating tissue (islet cells vs. kidney): Islet cells are pancreatic and clearly have different characteristics than renal cells. In clinical renal allografts, islet cells would not be used. Rather, renal cells or kidneys would be used.

The nature of the target (renal subcapsular space vs. kidney): In the previous study, the islet cells were transplanted into the renal subcapsular space. In the studies related to the present invention, the kidney itself was transplanted. Although the target tissue is similar, transplanting into the renal subcapsular space does not predict the response of transplanting an entire kidney. The prior observation, islet cells transplanted into the renal subcapsular space of CD103 knock-out mice were accepted indefinitely, does not predict the present observation, an entire kidney is accepted in animals with altered CD103.

Graft vasculature (non-vascularized vs. vascularized): Non-vascularized allografts and vascularized allografts do not necessarily elicit the same response in a host. The prior studies with non-vascularized allografts are not predictive of the present studies with vascularized allografts. Understanding the behavior of the vascularized allograft under experimental conditions is critical to predicting the behavior of clinical renal allograft rejection.

Given at least these differences, the present observations that an entire vascularized kidney was accepted in animals with altered CD103 could not have been predicted. The present observations are necessary for methods of preventing or treating clinical renal allograft rejections.

Methods of the Present Invention

The present invention relates to a method of preventing clinical renal allograft rejection, comprising administering to a patient in need thereof a therapeutically effective amount of a composition which affects the expression, activity, or other function of CD103. The patient may have received a clinical renal allograft, or may be a candidate for a clinical renal allograft. The composition may be a small molecule, a peptide, an antibody, or other composition. The compound may alter the expression of CD103, such as a transcription inhibitor. The compound may modulate a CD103 or CD8 precursor, effector, or effector precursor, for example. The compound may modulate a step in a pathway of CD103 expression, activation or modulation.

The compound may alter the activity or other functions of CD103. For example, the compound may alter the destruction of graft epithelial compartments by CD8 cells. The composition may comprise an antibody to CD103, such as OX-62, for example. The composition may comprise a modulator of a TNF-β receptor. The composition may be administered in combination with another compound. The composition may comprise a modulator of E-cadherin.

The methods of the present invention may be useful for the prevention or treatment of rejection of other allografts in other organ systems, such as pancreas, hepatocytes, alveoli, and other organ systems specialized epithelial layers, for example.

Methods of Identifying Therapeutic Compounds

Methods of identifying therapeutic compounds useful in the described methods of treatment and prevention are also included. These include methods of identifying binding partners of CD103, for example. These also include methods of identifying compounds that modulate the expression of a nucleic acid encoding CD103, for example. These also include methods of identifying agents that modulate at least one activity of CD103, for example.

Another embodiment of the present invention provides methods for use in isolating and identifying binding partners of proteins (e.g., CD103) described herein. In detail, a protein is mixed with a potential binding partner or an extract or fraction of a cell under conditions that allow the association of potential binding partners with the protein of the invention. After mixing, peptides, polypeptides, proteins or other molecules that have become associated with a protein of the invention are separated from the mixture. The binding partner that bound to the CD103 or fragment thereof can then be removed and further analyzed. To identify and isolate a binding partner, the entire protein, for instance the entire disclosed protein of SEQ ID NO:1 can be used. Alternatively, a fragment of the protein can be used.

As used herein, a cellular extract refers to a preparation or fraction which is made from a lysed or disrupted cell.

A variety of methods can be used to obtain cell extracts. Cells can be disrupted using either physical or chemical disruption methods. Examples of physical disruption methods include, but are not limited to, sonication and mechanical shearing. Examples of chemical lysis methods include, but are not limited to, detergent lysis and enzyme lysis. A skilled artisan can readily adapt methods for preparing cellular extracts in order to obtain extracts for use in the present methods.

Once an extract of a cell is prepared, the extract is mixed with the protein of the invention under conditions in which association of the protein with the binding partner can occur. A variety of conditions can be used, such as conditions that closely resemble conditions found in the cytoplasm of a human cell. Features such as osmolarity, pH, temperature, and the concentration of cellular extract used, can be varied to optimize the association of the protein with the binding partner.

After mixing under appropriate conditions, the bound complex is separated from the mixture. A variety of techniques can be utilized to separate the mixture. For example, antibodies specific to a protein of the invention can be used to immunoprecipitate the binding partner complex. Alternatively, standard chemical separation techniques such as chromatography and density/sediment centrifugation can be used.

After removal of non-associated cellular constituents found in the extract, the binding partner can be dissociated from the complex using conventional methods. For example, dissociation can be accomplished by altering the salt concentration or pH of the mixture.

To aid in separating associated binding partner pairs from the mixed extract, the CD103 can be immobilized on a solid support. For example, the CD103 can be attached to a nitrocellulose matrix or acrylic beads. Attachment of the CD103 to a solid support aids in separating peptide/binding partner pairs from other constituents found in the extract. The identified binding partners can be either a single protein or a complex made up of two or more proteins.

One preferred in vitro binding assay for CD103 would comprise a mixture of a CD103 polypeptide and one or more candidate binding targets or substrates. After incubating the mixture under appropriate conditions, one would determine whether CD103 or a polypeptide fragment thereof bound with the candidate substrate. For cell-free binding assays, one of the components usually comprises or is coupled to a label. The label may provide for direct detection, such as radioactivity, luminescence, optical or electron density, etc., or indirect detection such as an epitope tag, an enzyme, etc. A variety of methods may be employed to detect the label depending on the nature of the label and other assay components. For example, the label may be detected bound to the solid substrate or a portion of the bound complex containing the label may be separated from the solid substrate, and the label thereafter detected.

Another embodiment of the present invention provides methods for identifying agents that modulate the expression of a nucleic acid encoding a protein of the invention such as a protein having the amino acid sequence of SEQ ID NO:1. Such assays may utilize any available means of monitoring for changes in the expression level of the nucleic acids of the invention. As used herein, an agent is said to modulate the expression of a nucleic acid of the invention, for instance a nucleic acid encoding the protein having the sequence of (SEQ ID NO:1), if it is capable of up- or down-regulating expression of the nucleic acid in a cell (e.g., mRNA).

In one assay format, cell lines that contain reporter gene fusions between the open reading frame and any assayable fusion partner may be prepared. Numerous assayable fusion partners are known and readily available including the firefly luciferase gene and the gene encoding chloramphenicol acetyltransferase (Alam et al., 1990 Anal. Biochem. 188: 245-254). Cell lines containing the reporter gene fusions are then exposed to the agent to be tested under appropriate conditions and time. Differential expression of the reporter gene between samples exposed to the agent and control samples identifies agents which modulate the expression of a nucleic acid encoding the protein having the sequence of SEQ ID NO:1.

Additional assay formats may be used to monitor the ability of the agent to modulate the expression of a nucleic acid encoding a protein of the invention such as the protein of SEQ ID NO:1. For instance, mRNA expression may be monitored directly by hybridization to the nucleic acids of the invention. Cell lines are exposed to the agent to be tested under appropriate conditions and time and total RNA or mRNA is isolated by standard procedures such those disclosed in Sambrook et al. (1989).

Probes to detect differences in RNA expression levels between cells exposed to the agent and control cells may be prepared from the nucleic acids of the invention. It is preferable, but not necessary, to design probes which hybridize only with target nucleic acids under conditions of high stringency. Only highly complementary nucleic acid hybrids form under conditions of high stringency. Accordingly, the stringency of the assay conditions determines the amount of complementarity which should exist between two nucleic acid strands in order to form a hybrid. Stringency should be chosen to maximize the difference in stability between the probe:target hybrid and potential probe:non-target hybrids.

Probes may be designed from the nucleic acids of the invention through methods known in the art. For instance, the G+C content of the probe and the probe length can affect probe binding to its target sequence. Methods to optimize probe specificity are commonly available in Sambrook et al. (1989) or Ausubel et al. (Current Protocols in Molecular Biology, Greene Publishing Co., NY, 1995).

Hybridization conditions are modified using known methods, such as those described by Sambrook et al. (1989) and Ausubel et al. (1995) as required for each probe. Hybridization of total cellular RNA or RNA enriched for polyA RNA can be accomplished in any available format. For instance, total cellular RNA or RNA enriched for polyA RNA can be affixed to a solid support and the solid support exposed to at least one probe comprising at least one, or part of one of the sequences of the invention under conditions in which the probe will specifically hybridize. Alternatively, nucleic acid fragments comprising at least one, or part of one of the sequences of the invention can be affixed to a solid support, such as a porous glass wafer. The glass or silica wafer can then be exposed to total cellular RNA or polyA RNA from a sample under conditions in which the affixed sequences will specifically hybridize. Such glass wafers and hybridization methods are widely available, for example, those disclosed by Beattie (WO 95/11755). By examining for the ability of a given probe to specifically hybridize to an RNA sample from an untreated cell population and from a cell population exposed to the agent, agents which up or down regulate the expression of a nucleic acid encoding the protein having the sequence of SEQ ID NO:1 are identified.

Another embodiment of the present invention provides methods for identifying agents that modulate at least one activity of a CD103 protein, such as the protein having the amino acid sequence of SEQ ID NO:1. Such methods or assays may utilize any means of monitoring or detecting the desired activity. By “modulation” is meant the ability of an agent to agonize or antagonize at least one activity of CD103.

In one format, the relative amounts of a protein of the invention between a cell population that has been exposed to the agent to be tested compared to an un-exposed control cell population may be assayed. In this format, probes such as specific antibodies are used to monitor the differential expression of the protein in the different cell populations. Cell lines or populations are exposed to the agent to be tested under appropriate conditions and time. Cellular lysates may be prepared from the exposed cell line or population and a control, unexposed cell line or population. The cellular lysates are then analyzed with the probe.

For example, N- and C-terminal fragments of CD103 can be expressed in bacteria and used to search for proteins which bind to these fragments. Fusion proteins, such as His-tag or GST fusion to the N- or C-terminal regions of CD103 or a whole CD103 protein can be prepared. These fusion proteins can be coupled to Talon or Glutathione-Sepharose beads and then probed with cell lysates. Prior to lysis, the cells may be treated with rapamycin or other drugs which may modulate CD103 or proteins that interact with CD103. Lysate proteins binding to the fusion proteins can be resolved by SDS-PAGE, isolated and identified by protein sequencing or mass spectroscopy, as is known in the art.

Antibody probes are prepared by immunizing suitable mammalian hosts in appropriate immunization protocols using the peptides, polypeptides or proteins of the invention if they are of sufficient length, or, if desired, or if required to enhance immunogenicity, they can be conjugated to suitable carriers. Methods for preparing immunogenic conjugates with carriers such as bovine serum albumin (BSA), keyhole limpet hemocyanin (KLH), or other carrier proteins are well known in the art. In some circumstances, direct conjugation using, for example, carbodiimide reagents may be effective; in other instances linking reagents such as those supplied by Pierce Chemical Co., Rockford, Ill., may be desirable to provide accessibility to the hapten. The hapten peptides can be extended at either the amino or carboxy terminus with a cysteine residue or interspersed with cysteine residues, for example, to facilitate linking to a carrier. Administration of the immunogens is conducted generally by injection over a suitable time period and with use of suitable adjuvants, as is generally understood in the art. During the immunization schedule, titers of antibodies are taken to determine adequacy of antibody formation.

Anti-peptide antibodies can be generated using synthetic peptides. Synthetic peptides can be as small as 2-3 amino acids in length, but are preferably at least 3, 5, 10, or 15 or more amino acid residues long. Such peptides can be determined using programs such as DNAStar. The peptides are coupled to KLH using standard methods and can be immunized into animals such as rabbits. Polyclonal anti-CD103 peptide antibodies can then be purified, for example using Actigel beads containing the covalently bound peptide.

While the polyclonal antisera produced in this way may be satisfactory for some applications, for pharmaceutical compositions, use of monoclonal preparations is preferred. Immortalized cell lines which secrete the desired monoclonal antibodies may be prepared using the standard method of Kohler and Milstein or modifications which effect immortalization of lymphocytes or spleen cells, as is generally known. The immortalized cell lines secreting the desired antibodies are screened by immunoassay in which the antigen is the peptide hapten, polypeptide or protein. When the appropriate immortalized cell culture secreting the desired antibody is identified, the cells can be cultured either in vitro or by production in ascites fluid.

The desired monoclonal antibodies are then recovered from the culture supernatant or from the ascites supernatant. Fragments of the monoclonals or the polyclonal antisera which contain the immunologically significant portion can be used as agonists or antagonists of CD103 activity, as well as the intact antibodies. Use of immunologically reactive fragments, such as the Fab, scFV Fab′, of F(ab′)₂ fragments are often preferable, especially in a therapeutic context, as these fragments are generally less immunogenic than the whole immunoglobulin.

The antibodies or fragments may also be produced, using current technology, by recombinant means. Regions that bind specifically to the desired regions of receptor can also be produced in the context of chimeras with multiple species origin. Antibody reagents so created are contemplated for use diagnostically or as stimulants or inhibitors of CD103 activity.

Agents that are assayed in the above method can be randomly selected or rationally selected or designed. As used herein, an agent is said to be randomly selected when the agent is chosen randomly without considering the specific sequences involved in the association of the a protein of the invention alone or with its associated substrates, binding partners, etc. An example of randomly selected agents is the use a chemical library or a peptide combinatorial library, or a growth broth of an organism.

The agents of the present invention can be, as examples, peptides, small molecules, vitamin derivatives, as well as carbohydrates. A skilled artisan can readily recognize that there is no limit as to the structural nature of the agents of the present invention.

The peptide agents of the invention can be prepared using standard solid phase (or solution phase) peptide synthesis methods, as is known in the art. In addition, the DNA encoding these peptides may be synthesized using commercially available oligonucleotide synthesis instrumentation and produced recombinantly using standard recombinant production systems. The production using solid phase peptide synthesis is necessitated if non-nucleic acid-encoded amino acids are to be included. Another class of agents of the present invention are antibodies immunoreactive with critical positions of proteins of the invention. Antibody agents are obtained by immunization of suitable mammalian subjects with peptides, containing as antigenic regions, those portions of the protein intended to be targeted by the antibodies.

The present invention further contemplates therapeutic compositions useful in practicing the therapeutic methods of this invention. A subject therapeutic composition includes, in admixture, a pharmaceutically acceptable excipient (carrier) and one or more of the compounds described herein as an active ingredient.

The preparation of therapeutic compositions which contain polypeptides, analogs or active fragments as active ingredients is well understood in the art. Typically, such compositions are prepared as injectables, either as liquid solutions or suspensions, however, solid forms suitable for solution in, or suspension in, liquid prior to injection can also be prepared. The preparation can also be emulsified. The active therapeutic ingredient is often mixed with excipients which are pharmaceutically acceptable and compatible with the active ingredient. Suitable excipients are, for example, water, saline, dextrose, glycerol, ethanol, or the like and combinations thereof. In addition, if desired, the composition can contain minor amounts of auxiliary substances such as wetting or emulsifying agents, pH buffering agents which enhance the effectiveness of the active ingredient.

A compound can be formulated into the therapeutic composition as neutralized pharmaceutically acceptable salt forms. Pharmaceutically acceptable salts include the acid addition salts (formed with the free amino groups of the polypeptide or antibody molecule) and which are formed with inorganic acids such as, for example, hydrochloric or phosphoric acids, or such organic acids as acetic, oxalic, tartaric, mandelic, and the like. Salts formed from the free carboxyl groups can also be derived from inorganic bases such as, for example, sodium, potassium, ammonium, calcium, or ferric hydroxides, and such organic bases as isopropylamine, trimethylamine, 2-ethylamino ethanol, histidine, procaine, and the like.

The therapeutic compositions are conventionally administered intravenously, as by injection of a unit dose, for example. The term “unit dose” when used in reference to a therapeutic composition of the present invention refers to physically discrete units suitable as unitary dosage for humans, each unit containing a predetermined quantity of active material calculated to produce the desired therapeutic effect in association with the required diluent; i.e., carrier, or vehicle.

The compositions are administered in a manner compatible with the dosage formulation, and in a therapeutically effective amount. The quantity to be administered depends on the subject to be treated, capacity of the subject's immune system to utilize the active ingredient, and degree of modulation desired. Precise amounts of active ingredient required to be administered depend on the judgment of the practitioner and are peculiar to each individual. However, suitable dosages may range from about 0.001 to 30, preferably about 0.01 to about 25, and more preferably about 0.1 to 20 milligrams of active ingredient per kilogram body weight of individual per day and depend on the route of administration. Suitable regimes for initial administration and booster shots are also variable, but are typified by an initial administration followed by repeated doses at one or more hour intervals by a subsequent injection or other administration. Alternatively, continuous intravenous infusion sufficient to maintain concentrations of ten nanomolar to ten micromolar in the blood are contemplated.

The therapeutic compositions may further include an effective amount of the compound and one or more of the following active ingredients: an antibiotic, a steroid, and the like.

Animal Model of Renal Allograft Rejection

The present invention also relates to an animal model of renal allograft rejection. The animal model may involve transplantation of vascularized renal allografts into SCID mouse recipients. The SCID mouse recipients may be modified to be able to reject allografts. These modifications may involve the transfer of purified CD8+ T cells from wild type mice, or other mice with CD8+ T cells. The animal model of renal allograft rejection will be useful in methods of analyzing allograft rejection. This mouse model may be compared to other mouse allograft recipients. For example, allograft rejection in this mouse model may be compared to allograft rejection in a SCID mouse modified by the transfer of purified CD8+ T cells from a CD103 knockout mice.

### EXAMPLES

CD103, a T-cell integrin conferring specificity for the epithelial ligand E-cadherin, defines a major subset of CD8 effectors that infiltrate the graft renal tubules during clinical renal allograft rejection. CD103 knockout mice fail to reject epithelial allografts (pancreatic islets). Experiments described herein involve a rat model of vascularized renal transplantation. Unexpectedly, CD8 cells isolated from rat renal allografts undergoing unmodified acute rejection did not express significant levels of CD103. However, treatment of recipients with a brief course of CsA resulted in progressive accumulation of CD103+CD8+ cells in the graft. Consistent with the known clinical rejection scenario, CD103+CD8+ cells in such grafts exhibited a T effector phenotype and intercalated the graft tubular epithelium. Importantly, the frequency of CD103+CD8+ effectors in rejecting allografts correlated closely with the extent of tubulitis and destruction of the graft tubular epithelium. Treatment with non-depleting anti-CD103 mAb inhibited accumulation of CD8 cells in the graft renal tubules and attenuated rejection pathology. Together, these data documented a critical role for CD103+CD8+ effectors in destruction of the graft renal tubules during rejection of vascularized renal allografts. These experiments lead to the present invention, which relates to a method of preventing clinical renal allograft rejection, comprising administering to a patient in need thereof a therapeutically effective amount of a composition which affects the expression, activity, or other function of CD103.

Materials and Methods

Animals. Inbred male Brown Norway (BN) rats (200˜250 g) and Lewis (LEW) rats (200˜250 g) were supplied by Harlan Sprague-Dawley, Indianapolis, Ind. BN rats were used as kidney donors and Lewis rats were used as kidney recipients of renal allografts.

Surgical procedure. Orthotopic kidney transplants between BN donors and LEW recipients (or LEW to LEW as control of homogenic transplantation) were performed under the general anesthesia by inhalation of Halothane (Halocarbon, River Edge). The left donor kidney, ureter and patch of the bladder were isolated and removed en bloc, then flushed with 500 U/ml Heparin saline solution. After removal of recipient left kidney, the artery and vein of renal allograft were anastomosed end to end with the same vessels of recipient. The bladder patch of allograft was anastomosed to the bladder of recipient. The right native kidney of recipient was removed before closing the abdomen. Blood from the tail was used to measure the serum creatinine of recipient in different time point after transplantation. Heterotopic heart transplants from BN donors to LEW recipients were performed as reference.

Experimental Groups.

A) Classical acute rejection model: Recipient did not receive any immunosuppressant (n=4) after transplantation.

B) Cyclosporine delayed rejection model (n=13): recipient rats received Cyclosporine (5 mg/kg/day, im, Sandoz Pharmaceuticals, East Hanover, N.J.) for 10 days after transplantation to reverse an early rejection episode. All rats recovered and went on to survive. Allografts were harvested for FACS analyses and immunohistochemistry from different time point from 3 weeks to 12 weeks after transplantation.

C) Cyclosporine isografts control (n=4): kidney transplantations from LEWIS rats to LEWIS rats were performed and the allografts were harvested at 4 weeks post transplantation.

D) Late acute rejection model (n=5): in 5 week after Cyclosporine treated, 200 million spleen cells were IP injected into LEWIS recipients to induce late acute rejection.

E) Anti-CD103 blocking treatment (n=5): 2 days later after Cyclosporine injection, the recipients were injected with anti-CD103 antibody OX62 (BioExpress) every other day for continued 8 doses (3.5 mg/dose). The grafts were harvest at 4 weeks after transplantation. Control rats were injected with isotype IgG1 in the same dose.

Antibodies. For IHC, mouse anti rat CD103 (OX62) and anti rat CD8 mAbs (OX8) were purchased from Pharmingen (San Diego, Calif.), mouse anti human E-Cadherin mAbs were purchased from BD Transduction Laboratories (San Diego, Calif.), reactivity with rat. For FACS, FITC-conjugated mouse anti-rat CD103 mAbs (OX62) were purchased from Serotec (Oxford, England). Biotin-conjugated mouse anti-rat CD11a mAbs were purchased from Caltag (Burlingame, Calif.). Mouse mAbs PE-conjugated anti-rat CD8a, Biotin-conjugated anti-rat CD2, CD3, CD8b, CD44, CD49d, CD62L, αβTCR were purchased from Pharmingen (San Diego, Calif.).

Histology and Immunohistochemistry. Parts of Specimens were fixed in 10% phosphate-buffered formalin, routinely processed, and embedded in paraffin. 5 μm sections were stained with Hematoxylin-Eosin, PAS for general evaluation. Parts of Specimens were embedded in Tissue-Tek O.C.T (Sacura Finetec, Torrance, Calif.) and immediately frozen in liquid nitrogen. 6 μm cryostat sections were fixed in acetone and used for staining of CD103 and E-Cadherin.

CD8: The detection of CD8 was performed on 5 μm Paraffin Sections. The sections were incubated with primary anti CD8 mAb (0.5 μg/ml, 4° C. overnight), biotinylated goat anti mouse IgG and Streptavidin/Horse-radish peroxidase (Vector). The substrate is DAB (Vector). All sections for IHC were quenched of endogenous peroxidase activity by treatment with PBS containing 0.3% Hydrogen Peroxide.

Double staining of CD103 and E-Cadherin: After incubation with 3% normal goat serum and treatment with Biotin/Avidin Blocking Kit (Vector Laboratories, CA), the sections were incubated with anti-rat CD103 mAb (6 μg/ml) at 37° C. for one hour. Then added biotinylated goat anti mouse Immunoglobulin (multiple adsorbed, 5 μg/ml, Pharmingen) and Avidin DH:Biotinylated horse-radish peroxidase (Vectastain Elite ABC kit, Vector). The peroxidase label was developed using DAB (Vector) to detect CD103. Subsequently, sections were incubated with 3% horse serum, anti human E-Cadherin (10 μg/ml, 4° C. overnight), biotinylated horse anti mouse IgG (H+ L) (rat adsorbed, 5 μg/ml, Vector) and Avidin DH: biotinylated alkaline phophatase (Vectastain ABC-AP Kit, Vector). The substrate was Vector Red, with levamisole to inhibit endogenous alkaline phosphatase activity.

Flow Cytometry. Graft infiltrating lymphocytes (GIL) were isolated from allografts as described previously (a). Briefly, cortical pieces were rinsed to remove contaminating peripheral blood, then incubated for 30 min in DMEM/F12 (50:50) containing 0.1% collagenase (Type IV, Worthington, Freehold, N J), 0.1% soybean trypsin inhibitor (Sigma), and 0.01% DNase I (Boehringer Mannheim, Indianapolis, Ind.). Following vigorous agitation, the resulting cell suspension was centrifuged on Lympholyte-Rat (Cedarlane, Hornby, Ontario, Canada) to isolate lymphocytes. Lymphocytes from drainage lymphonote of graft were isolated by gentle grinding by glasses and adjusted into the same number with GIL. Freshly isolated GIL were stained for three-color Flow cytomery using anti-CD103-FITC and anti-CD8a-PE in conjunction with Cy-chrome-conjugated mAbs to rat CD2, CD3, CD8b, CD11a, CD49d and αβTCR. Mouse IgG1-FITC (Serotec) was as an isotype control of anti-CD103-FITC. After staining, cells were fixed with 0.5% paraformaldehyde and 50,000˜100,000 cells were analyzed using a FACScan (Becton Dickinson). Lymphocyte populations were gated by forward scatter/side scatter analysis to exclude dead cells and non-lymphocytes. WinMDI version2.8 developed by Dr. Joseph Trotter (Scripps Institute, San Diego, Calif.) was used for analysis and graphical display of flow cytometry data. Percent positive cells for a given marker and quadrant settings were based on cutoff points chosen to exclude >99% of the negative control population.

### Example 1

Low expression of CD103 in unmodified rejection of vascularized renal allografts

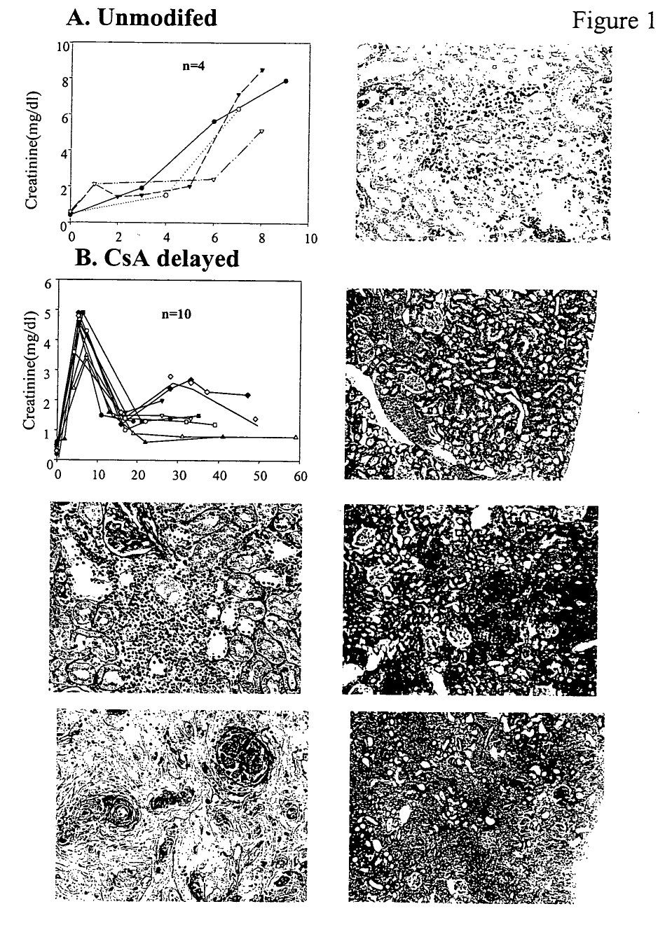

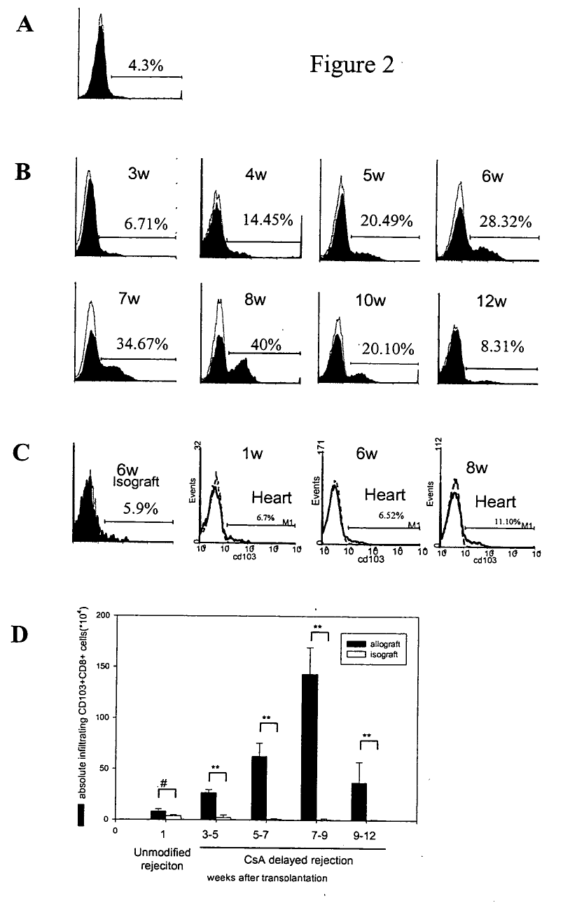

To assess the role of CD103+CD8+ effectors in rejection of vascularized renal allografts, first it was determined if CD103 is significantly expressed by CD8 effector populations that infiltrate rejecting renal allografts. For these studies, fully MHC-disparate BN kidneys were transplanted into LEW hosts. As shown in FIG. 1A, renal allografts in this strain combination were rapidly rejected as evidenced by the abrupt rise in serum creatinine (MST=7.8 days) accompanied by severe arteritis and transmural inflammatory infiltration, fibrinoid change, and interstitial hemorrhage (FIG. 1B), graded type IIIv3 by the Banff97 classification system. Importantly, however, there was a conspicuous absence of lymphocytic infiltration into the renal tubules (tubulitis), the sine qua non of clinical renal allograft rejection. Moreover, as shown in FIG. 2A, CD8 cells that infiltrated allografts at the time of rejection showed only low expression of (<8%) of CD103. Similar results were obtained for a variety of other rat strain combinations.

### Example 2

Progressive Accumulation of CD103+CD8+ Effectors in Renal Allografts Undergoing CsA Delayed Rejection

Clinical observational studies indicate that CD103+CD8+ effectors are most abundant in renal allografts undergoing late rejection episodes (i.e., acute-on-chronic rejection). To model this rejection scenario, the above model was modified by administering a brief course of CsA (10 days at 10 mg/kg/day) to LEW recipients of BN allografts. As shown in FIG. 1B, such grafts exhibited a brief elevation in serum creatinine levels which returned to normal and remained stable for >60 days. Histological analyses (FIG. 1B) revealed that grafts in this model undergo rejection episodes highly similar to the clinical situation with massive infiltration of mononuclear cells into graft interstitium accompanied by severe tubulitis. Moreover, grafts in this model went on to develop tubular atrophy and interstitial fibrosis (FIG. 1B), cardinal features of clinical chronic allograft nephropathy.

### Example 3

Characterization of CD103+CD8+ cells that Infiltrate Renal Allografts Undergoing CsA Delayed Rejection

As shown in FIG. 2B, FACS analyses of lymphocytes that infiltrated renal allografts undergoing CsA-delayed rejection revealed a progressive accumulation of CD103+CD8+ cells from 3 weeks through 8 weeks post transplantation. By 8 weeks post transplantation, 40% of graft infiltrating CD8+ cells were CD103 positive. In contrast, CD8 cells that infiltrated renal isografts or vascularized heart allografts (FIG. 2C) did not express significant levels of CD103 at any time point. The latter data indicate that CsA treatment by itself is not sufficient for accumulation of CD103+CD8+ cells at the graft site. As shown in FIG. 2D, the absolute number of CD103+CD8+ cells in the graft steadily increased from the time of CsA withdrawal through ˜8 weeks then declined sharply by 12 weeks post-transplantation.

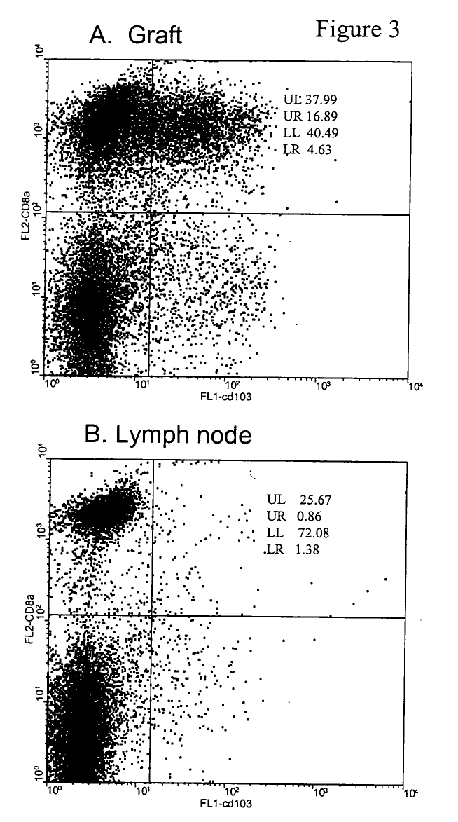

As shown in FIG. 3A, the vast majority (˜80%) of CD103+cells in the graft were CD8+ cells with little expression by non-CD8 cells. This distribution is highly similar to that previously described for rejecting clinical renal allografts and mouse pancreatic islet allografts transplanted into the renal subcapsule. As shown in FIG. 3B, CD8 cells in the renal lymph node of renal allograft recipients were almost completely devoid of CD103 expression, suggesting that CD103+CD8+ cells are sequestered at the graft site.

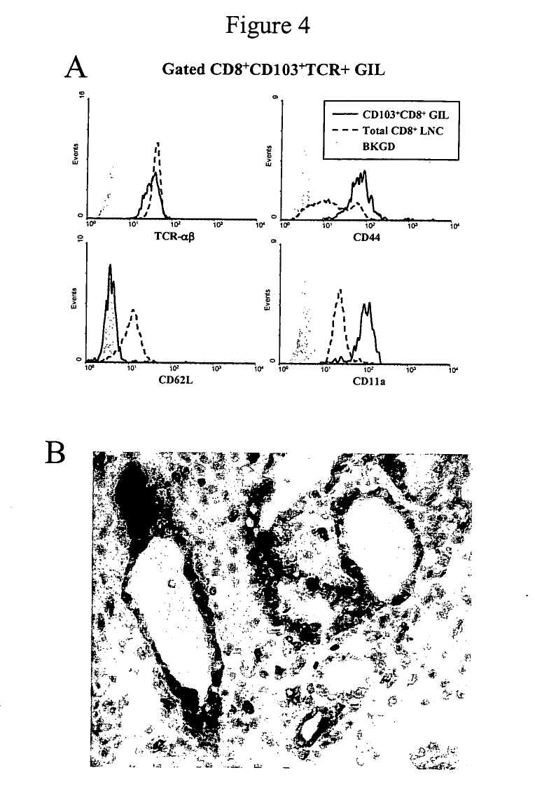

FIG. 4A shows that CD103+CD8+ cells that infiltrate renal allografts were CD44^{hi}, TCR-αβ⁺, CD11 a^{hi}, CD62L^{lo}, and thus exhibit a classic CD8 T effector phenotype. To determine the spatial relationship between CD103+CD8+ effectors and the graft tubular epithelium, double staining for CD103 and its ligand-Cadherin was performed on the cryostat sections of specimens with severe tubulitis. A subset of CD103+CD8+ effectors was intimately associated with the renal tubular epithelium which stains brightly for E-cadherin (FIG. 4B).

### Example 4

Role of CD103+CD8+ Effectors with Progression of Renal Allograft Rejection

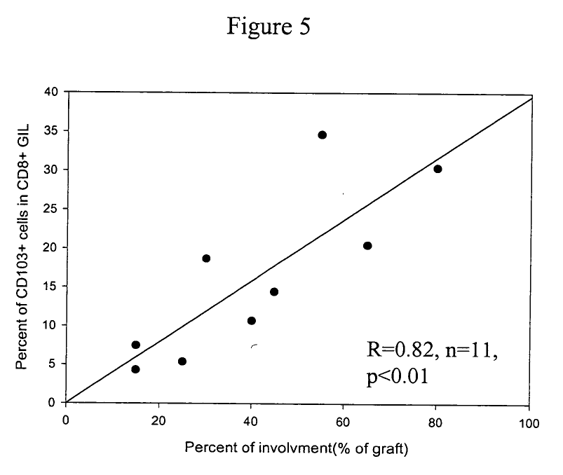

As shown in FIG. 5, the abundance of CD103+CD8+ effectors correlated closely (R=0.82) with the progression of graft destruction. Thus, a linear relationship exists between the percentage of CD8 effectors expressing CD103 and the proportion of the graft involved in the rejection process (R=0.82). These data indicated that CD103+CD8+ effectors play a critical role in the progression of tubulitis and graft destruction.

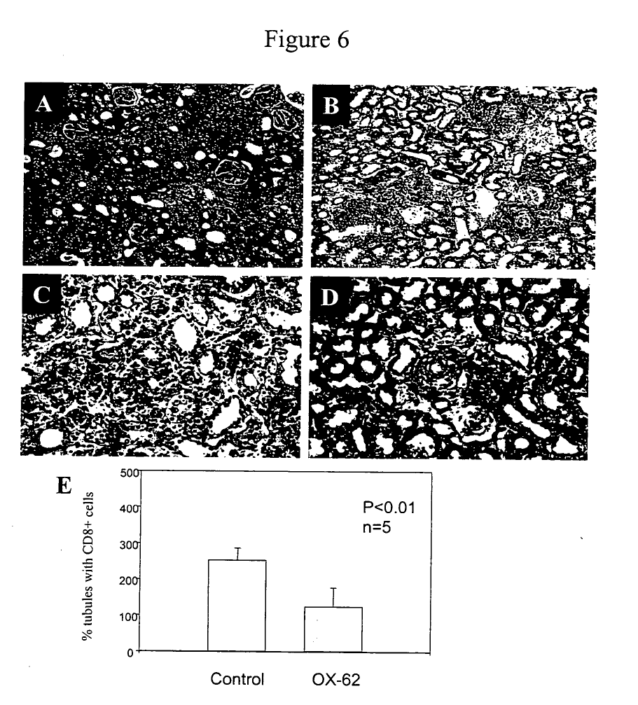

The capacity of anti-CD103 mAb to prevent graft destruction was assessed. In these experiments, a group of Lew recipients of BN renal allografts (n=4) was treated with a brief course of CsA (days 1-10), then treated with the anti-rat CD103 mAb, OX-62, from post-operative day 12 through 28 (8 doses of 3.5 mg i.p. every other day). A control cohort (n=4) received an isotype matched negative control mAb but otherwise was treated identically to the experimental group. As shown in FIG. 6, grafts from rats treated with OX-62 (FIG. 6B) showed dramatically reduced levels of tubulitis and tubular destruction of rejection grade as compared those from control rats (FIG. 6A). Immunostaining for CD8+ cells was performed to assess the extent to which CD8 cells accumulated in the tubules in the two groups. As shown in FIGS. 6C and 6D, OX-62 dramatically reduced accumulation of CD8+ T cells in the graft renal tubules. A quantitative analysis of the relative abundance of CD8+ cells infiltrating the graft renal tubules in the two groups confirmed that these differences are highly significant (FIG. 6E).

### Example 5

A simplified mouse model of renal allograft rejection that involves transplantation of vascularized renal allografts into SCID mouse recipients was developed. SCID mice entirely lack T cells and so can not reject the allografts. To elicit rejection in these mice, purified CD8+ T cells (CD8 cells) from either wild type mice or from CD103 knockout mice were transferred. The transferred wildtype CD8 cells became activated, infiltrated the graft renal tubules, and rapidly mediated graft destruction. In marked contrast, CD8 cells from CD103 knockout mice failed to penetrate the graft renal tubules remaining sequestered in the graft interstitium, and did not mediate graft destruction. These data clearly established that CD103 expression is required for destruction of the graft renal tubules by CD8+ T cells during rejection of vascularized renal allografts.

### Example 6

In Vitro Capacity of OX62-SAP To Deplete CD103+CD8+ Cells

Saporin is a potent inhibitor of cellular protein synthesis which induces death by apoptosis when internalized following antibody binding. The toxin Saporin was conjugated to anti-rat CD103 monoclonal antibody OX-62 to create the CD103 immunotoxin “OX62-SAP.”

CD103+CD8+ cells were generated by in vitro stimulation of spleen cells from normal Lewis rats with the mitogen Concanavalin A in the presence of rTGF-b1. OX62-SAP was then added at day 7 of culture, and cells were harvested at day 10 for analysis. FACS analyses to determine the percentage of CD8+ were performed. The total number of CD8+ cells in each culture was computed as the product of total cells in each culture times the percentage of CD8+ cells, as determined by FACS.

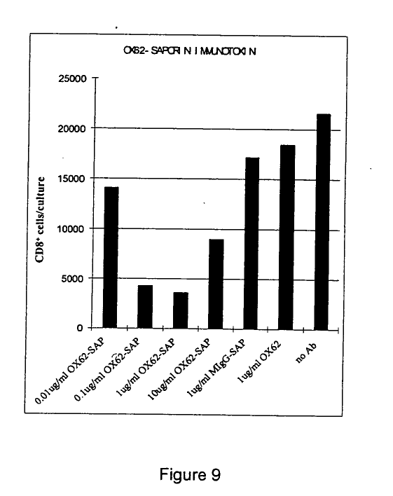

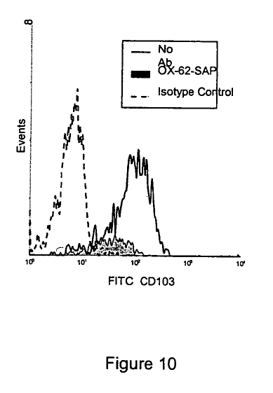

As shown in FIG. 9, OX62-SAP was highly-effective in depleting CD103+CD8+ cells from culture. In contrast, IgG1 conjugated to Saporin was ineffectual in this regard, as was unconjugated OX-62. Representative FACS plots are shown in FIG. 10.

### Example 7

In Vivo Capacity of OX62-SAP to Deplete CD103+CD8+ Cells

OX62-SAP was injected intravenously into normal rats. FACS analyses were then used to determine the number of CD8+ cells remaining in the intestinal intraepithelial lymphocyte (iIEL) compartment. The iIEL of normal rats contains a large population of CD8+ T cells, almost all (more than 95%) of which express high levels of CD103.

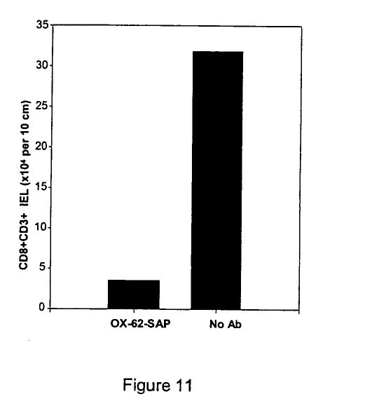

As shown in FIG. 11, the intravenous treatment by OX62-SAP dramatically depleted CD8+ T cells (CD8+ CD3+cells) from the iIEL compartment as compared to the mock treated controls. Thus, these data document the capacity of OX62-SAP to deplete CD103+CD8+ cells in vivo.

While the invention has been described and illustrated herein by references to various specific material, procedures and examples, it is understood that the invention is not restricted to the particular material combinations of material, and procedures selected for that purpose. Numerous variations of such details can be implied as will be appreciated by those skilled in the art.

The following is a list of documents related to the above disclosure and particularly to the experimental procedures and discussions. The following documents, as well as any documents referenced in the foregoing text, should be considered as incorporated by reference in their entirety.

### REFERENCES

1. Gjertson D W. 1999. Determinants of long-term survival of adult kidney transplants: a 1999 UNOS update. Clin Transplants: 341-52
2. Cai J, Gjertson D W, Terasaki P. 2002. Maintenance immunosuppressive treatment and graft half-life. Clin Transplants: 359-66
3. Matas A J, Gillingham K J, Payne W D, Najarian J S. 1994. The impact of an acute rejection episode on long-term renal allograft survival (t1/2). Transplantation 57: 857-9
4. Banchereau J, Steinman R M. 1998. Dendritic cells and the control of immunity. Nature 392: 245-52.
5. Butcher E C, Picker L J. 1996. Lymphocyte homing and homeostasis. Science 272: 60-6.
6. Basadonna G P, Matas A J, Gillingham K J, Payne W D, Dunn D L, Sutherland D E, Gores P F, Gruessner R W, Najarian J S. 1993. Early versus late acute renal allograft rejection: impact on chronic rejection. Transplantation 55: 993-5
7. Humar A, Kerr S, Gillingham K J, Matas A J. 1999. Features of acute rejection that increase risk for chronic rejection. Transplantation 68: 1200-3
8. Lindholm A, Ohiman S, Albrechtsen D, Tufveson G, Persson H, Persson N H. 1993. The impact of acute rejection episodes on long-term graft function and outcome in 1347 primary renal transplants treated by 3 cyclosporine regimens. Transplantation 56: 307-15
9. Sallusto F, Lenig D, Forster R, Lipp M, Lanzavecchia A. 1999. Two subsets of memory T lymphocytes with distinct homing potentials and effector functions. Nature 401: 708-12
10. van Stipdonk M J, Lemmens E E, Schoenberger S P. 2001. Naive CTLs require a single brief period of antigenic stimulation for clonal expansion and differentiation. Nat Immunol 2: 423-9
11. Topham D J, Tripp R A, Doherty P C. 1997. CD8+ T cells clear influenza virus by perforin or Fas-dependent processes. J Immunol 159: 5197-200.
12. Barth R J, Jr., Mule J J, Spiess P J, Rosenberg S A. 1991. Interferon gamma and tumor necrosis factor have a role in tumor regressions mediated by murine CD8+ tumor-infiltrating lymphocytes. J Exp Med 173: 647-58.
13. Diamond A S, Gill R G. 2000. An essential contribution by IFN-gamma to CD8+ T cell-mediated rejection of pancreatic islet allografts. J Immunol 165: 247-55.
14. Cocchi F, DeVico A L, Garzino-Demo A, Arya S K, Gallo R C, Lusso P. 1995. Identification of RANTES, MIP-1 alpha, and MIP-1 beta as the major HIV-suppressive factors produced by CD8+ T cells. Science 270: 1811-5.
15. Kim J J, Nottingham L K, Sin J I, Tsai A, Morrison L, Oh J, Dang K, Hu Y, Kazahaya K, Bennett M, Dentchev T, Wilson D M, Chalian A A, Boyer J D, Agadjanyan M G, Weiner D B. 1998. CD8 positive T cells influence antigen-specific immune responses through the expression of chemokines. J Clin Invest 102: 1112-24.
16. Sabek O, Dorak M T, Kotb M, Gaber A O, Gaber L. 2002. Quantitative detection of T-cell activation markers by real-time PCR in renal transplant rejection and correlation with histopathologic evaluation. Transplantation 74: 701-7
17. Dugre F J, Gaudreau S, Belles-Isles M, Houde I, Roy R. 2000. Cytokine and cytotoxic molecule gene expression determined in peripheral blood mononuclear cells in the diagnosis of acute renal rejection. Transplantation 70: 1074-80
18. Pascoe M D, Marshall S E, Welsh K I, Fulton L M, Hughes D A. 2000. Increased accuracy of renal allograft rejection diagnosis using combined perforin, granzyme B, and Fas ligand fine-needle aspiration immunocytology. Transplantation 69: 2547-53
19. Strehlau J, Pavlakis M, Lipman M, Maslinski W, Shapiro M, Strom T B. 1996. The intragraft gene activation of markers reflecting T-cell-activation and -cytotoxicity analyzed by quantitative RT-PCR in renal transplantation. Clin Nephrol 46: 30-3
20. Vasconcellos L M, Schachter A D, Zheng X X, Vasconcellos L H, Shapiro M, Harmon W E, Strom T B, Schachter D. 1998. Cytotoxic lymphocyte gene expression in peripheral blood leukocytes correlates with rejecting renal allografts. Transplantation 66: 562-6
21. Li B, Hartono C, Ding R, Sharma V K, Ramaswamy R, Qian B, Serur D, Mouradian J, Schwartz J E, Suthanthiran M. 2001. Noninvasive diagnosis of renal-allograft rejection by measurement of messenger RNA for perforin and granzyme B in urine. N Engl J Med 344: 947-54
22. Robertson H, Wheeler J, Kirby J A, Morley A R. 1996. Renal allograft rejection—in situ demonstration of cytotoxic intratubular cells. Transplantation 61: 1546-9.
23. Meehan S M, McCluskey R T, Pascual M, Preffer F I, Anderson P, Schlossman S F, Colvin R B. 1997. Cytotoxicity and apoptosis in human renal allografts: identification, distribution, and quantitation of cells with a cytotoxic granule protein GMP-17 (TIA-1) and cells with fragmented nuclear DNA. Lab Invest 76: 63949.
24. Racusen L C, Solez K, Colvin R B, Bonsib S M, Castro M C, Cavallo T, Croker B P, Demetris A J, Drachenberg C B, Fogo A B, Furness P, Gaber L W, Gibson I W, Glotz D, Goldberg J C, Grande J, Halloran P F, Hansen H E, Hartley B, Hayry P J, Hill C M, Hoffman E O, Hunsicker L G, Lindblad A S, Yamaguchi Y, et al. 1999. The Banff 97 working classification of renal allograft pathology. Kidney Int 55: 713-23.
25. Steinmuller D. 1985. Which T cells mediate allograft rejection? Transplantation 40: 229-33.
26. Janssen E M, Lemmens E E, Wolfe T, Christen U, von Herrath M G, Schoenberger S P. 2003. CD4+ T cells are required for secondary expansion and memory in CD8+ T lymphocytes. Nature 421: 852-6
27. Coffman T, Geier S, Ibrahim S, Griffiths R, Spurney R, Smithies O, Koller B, Sanfilippo F. 1993. Improved renal function in mouse kidney allografts lacking MHC class I antigens. J Immunol 151: 425-35
28. Russell P S, Chase C M, Colvin R B, Plate J M. 1978. Induced immune destruction of long-surviving, H-2 incompatible kidney transplants in mice. J Exp Med 147: 1469-86
29. Zhang Z, Zhu L, Quan D, Garcia B, Ozcay N, Duff J, Stiller C, Lazarovits A, Grant D, Zhong R. 1996. Pattern of liver, kidney, heart, and intestine allograft rejection in different mouse strain combinations. Transplantation 62: 1267-72
30. Bickerstaff A A, Wang J J, Pelletier R P, Orosz C G. 2001. Murine renal allografts: spontaneous acceptance is associated with regulated T cell-mediated immunity. J Immunol 167: 4821-7
31. Gracie J A, Bolton E M, Porteous C, Bradley J A. 1990. T cell requirements for the rejection of renal allografts bearing an isolated class I MHC disparity. J Exp Med 172: 1547-57
32. Larson R S, Springer T A. 1990. Structure and function of leukocyte integrins. Immunol Rev 114: 181-217
33. Cerf-Bensussan N, Jarry A, Brousse N, Lisowska-Grospierre B, Guy-Grand D, Griscelli C. 1987. A monoclonal antibody (HML-1) defining a novel membrane molecule present on human intestinal lymphocytes. Eur J Immunol 17: 1279-85.
34. Karecla P I, Bowden S J, Green S J, Kilshaw P J. 1995. Recognition of E-cadherin on epithelial cells by the mucosal T cell integrin alpha M290 beta 7 (alpha E beta 7). Eur J Immunol 25: 852-6.
35. Cepek K L, Shaw S K, Parker C M, Russell G J, Morrow J S, Rimm D L, Brenner M B. 1994. Adhesion between epithelial cells and T lymphocytes mediated by E-cadherin and the alpha E beta 7 integrin. Nature 372: 190-3.
36. Schon M P, Arya A, Murphy E A, Adams C M, Strauch U G, Agace W W, Marsal J, Donohue J P, Her H, Beier D R, Olson S, Lefrancois L, Brenner M B, Grusby M J, Parker C M. 1999. Mucosal T lymphocyte numbers are selectively reduced in integrin alpha E (CD103)-deficient mice. J Immunol 162: 6641-9.
37. Lefrancois L, Parker C M, Olson S, Muller W, Wagner N, Schon M P, Puddington L. 1999. The role of beta7 integrins in CD8 T cell trafficking during an antiviral immune response. J Exp Med 189: 1631-8.
38. Picker L J, Terstappen L W, Rott L S, Streeter P R, Stein H, Butcher E C. 1990. Differential expression of homing-associated adhesion molecules by T cell subsets in man. J Immunol 145: 3247-55
39. Hadley G A, Bartlett S T, Via C S, Rostapshova E A, Moainie S. 1997. The epithelial cell-specific integrin, CD103 (alpha E integrin), defines a novel subset of alloreactive CD8+ CTL. J Immunol 159: 3748-56.
40. Wang D, Yuan R, Feng Y, El-Asady R, Farber D L, Gress R E, Lucas P J, Hadley G A. 2004. Regulation of CD103 expression by CD8+ T cells responding to renal allografts. J Immunol 172: 214-21
41. Brew R, West D C, Burthem J, Christmas S E. 1995. Expression of the human mucosal lymphocyte antigen, HML-1, by T cells activated with mitogen or specific antigen in vitro. Scand J Immunol 41: 553-62
42. Hadley G A, Rostapshova E A, Gomolka D M, Taylor B M, Bartlett S T, Drachenberg C I, Weir M R. 1999. Regulation of the epithelial cell-specific integrin, CD103, by human CD8+ cytolytic T lymphocytes. Transplantation 67: 1418-25
43. Robinson P W, Green S J, Carter C, Coadwell J, Kilshaw P J. 2001. Studies on transcriptional regulation of the mucosal T-cell integrin alphaEbeta7 (CD103). Immunology 103: 146-54
44. Kilshaw P J, Murant S J. 1991. Expression and regulation of beta 7(beta p) integrins on mouse lymphocytes: relevance to the mucosal immune system. Eur J Immunol 21: 2591-7
45. Hadley G A, Charandee C, Weir M R, Wang D, Bartlett S T, Drachenberg C B. 2001. CD103+ CTL accumulate within the graft epithelium during clinical renal allograft rejection. Transplantation 72: 1548-55
46. Robertson H, Wong W K, Talbot D, Burt A D, Kirby J A. 2001. Tubulitis after renal transplantation: demonstration of an association between CD103+T cells, transforming growth factor beta1 expression and rejection grade. Transplantation 71: 306-13.
47. Leteurtre E, Copin M C, Labalette M, Noel C, Roumilhac D, Pruvot F R, Lecomte-Houcke M, Gosselin B, Dessaint J P. 2000. Negative immunohistochemical detection of CD103 (alphaEbeta7 integrin) in the infiltrates of acute rejection in liver and kidney transplantation. Transplantation 70: 227-9
48. Ding R, Li B, Muthukumar T, Dadhania D, Medeiros M, Hartono C, Serur D, Seshan S V, Sharma V K, Kapur S, Suthanthiran M. 2003. CD103 mRNA levels in urinary cells predict acute rejection of renal allografts. Transplantation 75: 1307-12
49. Feng Y, Wang D, Yuan R, Parker C M, Farber D L, Hadley G A. 2002. CD103 expression is required for destruction of pancreatic islet allografts by CD8(+) T cells. J Exp Med 196: 877-86
50. Gorelik L, Flavell R A. 2002. Transforming growth factor-beta in T-cell biology. Nat Rev Immunol 2: 46-53.
51. Wahl S M. 1994. Transforming growth factor beta: the good, the bad, and the ugly. J Exp Med 180: 1587-90.
52. Pardoux C, Asselin-Paturel C, Chehimi J, Gay F, Mami-Chouaib F, Chouaib S. 1997. Functional interaction between TGF-beta and IL-12 in human primary allogeneic cytotoxicity and proliferative response. J Immunol 158: 136-43.
53. Smyth M J, Strobl S L, Young H A, Ortaldo J R, Ochoa A C. 1991. Regulation of lymphokine-activated killer activity and pore-forming protein gene expression in human peripheral blood CD8+ T lymphocytes. Inhibition by transforming growth factor-beta. J Immunol 146: 3289-97.
54. Jain S, Mohamed M A, Sandford R, Furness P N, Nicholson M L, Talbot D. 2002. Sequential protocol biopsies from renal transplant recipients show an increasing expression of active TGF beta. Transpl Int 15: 630-4
55. Cuhaci B, Kumar M S, Bloom R D, Pratt B, Haussman G, Laskow D A, Alidoost M, Grotkowski C, Cahill K, Butani L, Sturgill B C, Pankewycz O G. 1999. Transforming growth factor-beta levels in human allograft chronic fibrosis correlate with rate of decline in renal function. Transplantation 68: 785-90
56. Suthanthiran M. 1997. Clinical application of molecular biology: a study of allograft rejection with polymerase chain reaction. Am J Med Sci 313: 264-7
57. Wahl S M. 1994. Transforming growth factor beta: the good, the bad, and the ugly. J Exp Med 180: 1587-90
58. McHugh R S, Whitters M J, Piccirillo C A, Young D A, Shevach E M, Collins M, Byrne M C. 2002. CD4(+)CD25(+) immunoregulatory T cells: gene expression analysis reveals a functional role for the glucocorticoid-induced TNF receptor. Immunity 16: 311-23
59. Huehn J, Siegmund K, Lehmann J C, Siewert C, Haubold U, Feuerer M, Debes G F, Lauber J, Frey O, Przybylski G K, Niesner U, De La Rosa M, Schmidt C A, Brauer R, Buer J, Scheffold A, Hamann A. 2004. Developmental Stage, Phenotype, and Migration Distinguish Naive- and Effector/Memory-like CD4+ Regulatory T Cells. J Exp Med 199: 303-13
60. Lehmann J, Huehn J, de la Rosa M, Maszyna F, Kretschmer U, Krenn V, Brunner M, Scheffold A, Hamann A. 2002. Expression of the integrin alpha Ebeta 7 identifies unique subsets of CD25+ as well as CD25− regulatory T cells. Proc Natl Acad Sci U S A 99: 13031-6
61. Banz A, Peixoto A, Pontoux C, Cordier C, Rocha B, Papiernik M. 2003. A unique subpopulation of CD4+ regulatory T cells controls wasting disease, IL-10 secretion and T cell homeostasis. Eur J Immunol 33: 2419-28
62. Chen W, Jin W, Hardegen N, Lei K J, Li L, Marinos N, McGrady G, Wahl S M. 2003. Conversion of peripheral CD4+ CD25− naive T cells to CD4+ CD25+regulatory T cells by TGF-beta induction of transcription factor Foxp3. J Exp Med 198: 1875-86
63. Rostapshova E A, Burns J M, Bartlett S T, Hadley G A. 1998. Integrin-mediated interactions influence the tissue specificity of CD8+ cytolytic T lymphocytes. Eur J Immunol 28: 3031-9
64. Roberts K, Kilshaw P J. 1993. The mucosal T cell integrin alpha M290 beta 7 recognizes a ligand on mucosal epithelial cell lines. EurJ Immunol 23: 1630-5
65. Higgins J M, Mandlebrot D A, Shaw S K, Russell G J, Murphy E A, Chen Y T, Nelson W J, Parker C M, Brenner M B. 1998. Direct and regulated interaction of integrin alphaEbeta7 with E-cadherin. J Cell Biol 140: 197-210
66. Wong W K, Robertson H, Carroll H P, Ali S, Kirby J A. 2003. Tubulitis in renal allograft rejection: role of transforming growth factor-beta and interleukin-15 in development and maintenance of CD103+ intraepithelial T cells. Transplantation 75: 505-14
67. Campbell D J, Butcher E C. 2002. Rapid acquisition of tissue-specific homing phenotypes by CD4(+) T cells activated in cutaneous or mucosal lymphoid tissues. J Exp Med 195: 135-41
68. Robertson H, Kirby J A. 2003. Post-transplant renal tubulitis: the recruitment, differentiation and persistence of intra-epithelial T cells. Am J Transplant 3: 3-10
69. Higgins J M, Mandlebrot D A, Shaw S K, Russell G J, Murphy E A, Chen Y T, Nelson W J, Parker C M, Brenner M B. 1998. Direct and regulated interaction of integrin alphaEbeta7 with E-cadherin. J Cell Biol 140: 197-210.
70. Sarnacki S, Begue B, Buc H, Le Deist F, Cerf-Bensussan N. 1992. Enhancement of CD3-induced activation of human intestinal intraepithelial lymphocytes by stimulation of the beta 7-containing integrin defined by HML-1 monoclonal antibody. Eur J Immunol 22: 2887-92.
71. Russell G J, Parker C M, Cepek K L, Mandelbrot D A, Sood A, Mizoguchi E, Ebert E C, Brenner M B, Bhan A K. 1994. Distinct structural and functional epitopes of the alpha E beta 7 integrin. Eur J Immunol 24: 2832-41.
72. Lefrancois L, Barrett T A, Havran W L, Puddington L. 1994. Developmental expression of the alpha IEL beta 7 integrin on T cell receptor gamma delta and T cell receptor alpha beta T cells. Eur J Immunol 24: 635-40.
73. Roberts K, Kilshaw P J. 1993. The mucosal T cell integrin alpha M290 beta 7 recognizes a ligand on mucosal epithelial cell lines. Eur J Immunol 23: 1630-5.
74. Rostapshova E A, Burns J M, Bartlett S T, Hadley G A. 1998. Integrin-mediated interactions influence the tissue specificity of CD8+ cytolytic T lymphocytes. Eur J Immunol 28: 3031-9.

1. A method of preventing or treating allograft rejection, comprising administering to a patient in need thereof a therapeutically-effective amount of a composition which affects the expression, activity, or other function of CD103.

2. The method of claim 1, wherein said composition comprises a small molecule, a peptide, or an antibody.

3. The method of claim 1, wherein said composition alters the expression of CD103.

4. The method of claim 3, wherein said composition inhibits the transcription of CD103.

5. The method of claim 1, wherein said composition modulates a CD103 or CD8 precursor, effector, or effector precursor.

6. The method of claim 1, wherein said composition modulates a step in a pathway of CD103 expression, activation, or modulation.

7. The method of claim 1, wherein said composition alters the activity or other functions of CD103.

8. The method of claim 7, wherein said composition alters the destruction of graft epithelial compartments by CD8 cells.

9. The method of claim 2, wherein said antibody is an antibody to CD103.

10. The method of claim 9, wherein said antibody is OX-62.

11. The method of claim 1, wherein said composition comprises a modulator of a TNF-β receptor.

12. The method of claim 1, wherein said composition comprises a modulator of E-cadherin.

13. The method of claim 1, wherein said allograft rejection is clinical renal allograft rejection.

14. The method of claim 1, wherein said allograft rejection occurs in the pancreas, hepatocytes, alveoli, or other organ systems' specialized epithelial layers.

15. A method for identifying agents which modulate the expression of a nucleic acid encoding a CD103 protein, comprising:
(a) providing a cell line which comprises a reporter gene fusion between the open reading frame encoding said CD 103 protein and an assayable fusion partner;
(b) exposing the cell line to the agent to be tested;
(c) determining the expression of the nucleic acid in the cell line so exposed; and
(d) comparing expression of the reporter gene in the cell line to the expression of the reporter gene in a cell line not exposed to the agent to be tested.

16. The method of claim 15, wherein said CD103 protein has the amino acid sequence of SEQ ID NO:1.

17. The method of claim 15, wherein said assayable fusion partner is the firefly luciferase gene or the chloramphenicol acetyltransferase gene.

18. The method of claim 15, wherein said expression is determined by measuring total RNA or mRNA levels.

19. A method for identifying an agent that modulates at least one activity of a CD103 protein, comprising:
(a) comparing the relative amount of said CD103 protein in a cell population that has been exposed to said agent to be tested to the amount of said CD103 protein in an unexposed control cell population.

20. The method of claim 19, wherein antibodies are used to monitor the differential expression of said CD103 protein.

21. The method of claim 19, wherein said agent is a peptide, small molecule, vitamin derivative, or carbohydrate.

22. An animal model of renal allograft rejection, comprising:
(a) Lewis rats that have been transplanted with fully MHC-disparate kidneys of Brown Norway rats.

23. The model of claim 22, wherein said Lewis rats receive 10 mg/kg/day of CsA for ten days following said transplantation.

24. The model of claim 23, wherein said Lewis rats receive eight doses of 3.5 mg i.p. of the anti-rat monoclonal antibody OX-62 from post-operative days 12-28.

25. A mouse model of renal allograft rejection, comprising:
(a) SCID mice into which purified CD8+ T cells from wild type mice have been transferred; and
(b) SCID mice into which purified CD8+ T cells from CD103 knockout mice have been transferred.


SEQUENCE LISTING (2 sequences)

SEQ ID NO: 1 - protein, 1179 aa, Homo sapiens
MWLFHTLLCIASLALLAAFNVDVARPWLTPKGGAPFVLSSLLHQDPSTNQTWLLVTSPRTKRTPGPLHRCSLVQDEILCHPVEHVPIPKGRHRGVTVVRSHHGVLICIQVLVRRPHSLSSELTGTCSLLGPDLRPQAQANFFDLENLLDPDARVDTGDCYSNKEGGGEDDVNTARQRRALEKEEEEDKEEEEDEEEEEAGTEIAIILDGSGSIDPPDFQRAKDFISNMMRNFYEKCFECNFALVQYGGVIQTEFDLRDSQDVMASLARVQNITQVGSVTKTASAMQHVLDSIFTSSHGSRRKASKVMVVLTDGGIFEDPLNLTTVINSPKMQGVERFAIGVGEEFKSARTARELNLIASDPDETHAFKVTNYMALDGLLSKLRYNIISMEGTVGDALHYQLAQIGFSAQILDERQVLLGAVGAFDWSGGALLYDTRSRRGRFLNQTAAAAADAEAAQYSYLGYAVAVLHKTCSLSYVAGAPQYKHHGAVFELQKEGREASFLPVLEGEQMGSYFGSELCPVDIDMDGSTDFLLVAAPFYHVHGEEGRVYVYRLSEQDGSFSLARILSGHPGFTNARFGFAMAAMGDLSQDKLTDVAIGAPLEGFGADDGASFGSVYIYNGHWDGLSASPSQRIRASTVAPGLQYFGMSMAGGFDISGDGLADITVGTLGQAVVFRSRPVVRLKVSMAFTPSALPIGFNGVVNVRLCFEISSVTTASESGLREALLNFTLDVDVGKQRRRLQCSDVRSCLGCLREWSSGSQLCEDLLLMPTEGELCEEDCFSNASVKVSYQLQTPEGQTDHPQPILDRYTEPFAIFQLPYEKACKNKLFCVAELQLATTVSQQELVVGLTKELTLNINLTNSGEDSYMTSMALNYPRNLQLKRMQKPPSPNIQCDDPQPVASVLIMNCRIGHPVLKRSSAHVSVVWQLEENAFPNRTADITVTVTNSNERRSLANETHTLQFRHGFVAVLSKPSIMYVNTGQGLSHHKEFLFHVHGENLFGAEYQLQICVPTKLRGLQVAAVKKLTRTQASTVCTWSQERACAYSSVQHVEEWHSVSCVIASDKENVTVAAEISWDHSEELLKDVTELQILGEISFNKSLYEGLNAENHRTKITVVFLKDEKYHSLPIIIKGSVGGLLVLIVILVILFKCGFFKRKYQQLNLESIRKAQLKSENLLEEEN

SEQ ID NO: 2 - protein, 1167 aa, Mus sp.
MKWLFHTLLCMASLKPQGAFNMDVDWAWVTALQPGAPAVLSSLLHLDPSNNQTCLLVARRSSNRNTAALYRCAISISPDEIACQPVEHICMPKGRYQGVTLVGNHNGVLVCIQVQARKFRSLNSELTGACSLLTPNLDLQAQAYFSDLEGFLDPGAHVDSGDYCRSKGGSTGEEKKSARRRRTVEEEDEEEDGTEIAIVLDGSGSIGPSDFQKAKNFISTMMRNFYEKCFECNFALVQYGAVIQTEFDLQESRDINASLAKVQSIVQVKEVTKTASAMQHVLDNIFIPSRGSRKKALKVMVVLTDGDIFGDPLNLTTVINSPKMQGVVRFAIGVGDRFKNNNTYRELKLIASDPKEAHTFKVTNYSALDGLLSKLQQRIVHMEGTVGDALQYQLAQTGFSAQILDKGQVLLGTVGAFNWSGGALLYSTQNGRGCFLNQTAKEDSRTVQYSYLGYSLAVLHKAHGISYVAGAPRHKLRGAVFELRKEDREEDAFVRRIEGEQMGSYFGSVLCPVDIDMDGTTDFLLVAAPFYHIRGEEGRVYVYQVPEQDASFSLAHTLSGHPGLTNSRFGFAMAAVGDINQDKFTDVAIGAPLEGFGAGDGASYGSVYIYNGHSGGLYDSPSQQIRASSVASGLHYFGMSVSGGLDFNGDGLADITVGSRDSAVVLRSRPVVDLTVSMTFTPDALPMVFIGKMDVNLCFEVDSSVVASEPGLREMFLNFTVDVDVTKQRQRLQCEDSSGCQSCLRKWNGGSFLCEHFWLISTEELCEEDCFSNITIKVTYEFQTSGGRRDYPNPTLDHYKEPSAIFQLPYEKDCKNKVFCIAEIQLTTNISQQELVVGVTKEVTMNISLTNSGEDSYMTNMALNYPRNLQFKKIQKPVSPDVQCDDPKPVASVLVMNCKIGHPILKRSSVNVSVTWQLEESVFPNRTADITVTISNSNEKSLARETRSLQFRHAFIAVLSRPSVMYMNTSQSPSDHKEFFFNVHGENLFGAVFQLQICVPIKLQDFQIVRVKNLTKTQDHTECTQSQEPACGSDPVQHVKEWHSVVCAITSNKENVTVAAEISVGHTKQLLRDVSELPILGEISFNKSLYEGLNAENHRTKITVIFLKEEETRSLPLIIGSSIGGLLVLVVIIAILFKCGFFKRKYQQLNLESTRRAQLKADSLLQD